(19) United States
(12) Patent Application Publication
(10) Pub. No.: US 2006/0258705 A1
(43) Pub. Date: Nov. 16, 2006

(54) Process for making crystalline donepezil hydrochloride monohydrate

(76) Inventor: Gerrit J. B. Ettema, Nijmegen (NL)

Correspondence Address: MARK R. BUSCHER;SYNTHON IP INC, 7130 HERITAGE VILLAGE PLAZA, STE 202, GAINESVILLE, VA, 20155, US

(21) Appl. No.: 11/377,399

(22) Filed: Mar. 17, 2006

Related U.S. Application Data
(60) Provisional application No. 60/662,546, filed on Mar. 17, 2005.

(51) Int. Cl. A61K 31/445

(52) U.S. Cl. 514/319

(57) ABSTRACT

Solvent mediated polymorphic transformations of crystalline donepezil hydrochloride monohydrate are reduced by using a C5-C8 hydrocarbon as a protecting liquid.

This application claims the benefit of priority under 35 U.S.C. § 119(e) from U.S. provisional application 60/662,546, filed Mar. 17, 2005, the entire contents thereof being incorporated herein by reference.

### BACKGROUND OF THE INVENTION

Donepezil, i.e. 1-benzyl-4-((5,6-dimethoxy-1-indanon)-2-yl)methylpiperidine of the general formula (1) 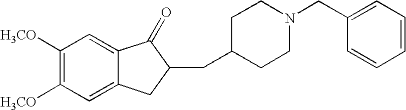
is a commercially marketed, pharmaceutically active substance useful for the treatment of, inter alia, senile dementia and in ameliorating Alzheimer's disease. The commercially marketed product is the hydrochloride salt of the compound (1). Donepezil hydrochloride is a white crystalline powder freely soluble in chloroform, soluble in water and in glacial acetic acid, slightly soluble in ethanol and acetonitrile and practically insoluble in ethyl acetate and in n-hexane.

Donepezil as well as donepezil hydrochloride was disclosed in EP 296560 (U.S. Pat. No. 4,895,841). Crystalline donepezil hydrochloride was provided in this document by a recrystallization from ethanol/isopropylether.

Donepezil hydrochloride in crystalline state is polymorphic. In a series of two patent applications WO 97-46526 and WO 97-46527, five polymorphs of crystalline donepezil hydrochloride, Forms I-V, were disclosed and characterized by specific X-ray diffraction patterns and IR spectra. Within the examination of the corresponding application at the EPO (EP 101 93 74), it was concluded that the Form I is identical with the original crystalline form provided in the earlier EP 296560/U.S. Pat. No. 4,895,841. The remaining Forms were considered new over the original patent disclosure.

Among the known polymorphs, the Form I is particularly important. Contrary to the other known forms, it has the unique characteristic that it comprises water in an amount equivalent to about 1:1 molar ratio (the WO 97-46527 reports the water content is within the range of 5.17 to 5.87%). While it is not entirely clear whether this water is bond crystalline water or not, for convenience this Form is referred to herein as crystalline donepezil hydrochloride monohydrate.

The crystalline donepezil hydrochloride monohydrate is not hygroscopic up to more than 90% humidity. As far as the chemical stability is concerned, it is comparably stable to other disclosed polymorphs. It can also be prepared by a simple crystallization procedure.

A variety of procedures have been proposed and/or used for the production of donepezil hydrochloride monohydrate. In general, methanol or ethanol is used as a solvent for recrystallization or as a salt forming reaction media followed by addition of a contra-solvent. For example, U.S. Pat. No. 4,895,841 teaches recrystallization of donepezil hydrochloride from ethanol/isopropylether mixture. WO 97-46527 lists 11 different variants for obtaining the Form I polymorph, which are consolidated below in the following list:

crystallizing donepezil hydrochloride from methanol or ethanol;

dissolving donepezil hydrochloride in methanol and adding diethylether, diisopropyl ether, t-butylmethyl ether, ethyl acetate or n-hexane to the solution;

dissolving donepezil hydrochloride in ethanol, followed by addition of tert.butyl methyl ether;

dissolving donepezil base in methanol, ethanol, tetrahydrofuran or acetonitrile and adding hydrochloric acid or hydrogen chloride to the solution;

dissolving donepezil base in ethanol, adding hydrochloric acid or hydrogen chloride and then diisopropylether or vice versa; and

dissolving donepezil base in methanol, adding hydrochloric acid/hydrogen chloride, followed by addition of tert.butyl methyl ether, diisopropyl ether or ethyl acetate.

However, crystalline donepezil hydrochloride monohydrate has been reported as being susceptible to solvent mediated conversions to different polymorphic forms. For instance, WO 97-46527 teaches that in the process of dissolving donepezil in ethanol, followed by addition of hydrochloric acid or hydrogen chloride, and then adding isopropyl ether, any of Forms (I), (II), or (III) can be made, depending upon the lag time between crystallization and filtering off the crystals. Crystalline donepezil hydrochloride Form (I)—the monohydrate—is obtainable only when the formed crystals are filtered immediately after the crystallization. When the lag time between the precipitation and filtration is between 10-60 minutes, the polymorph (II) is formed. When the filtration of the crystals occurs more than one hour after precipitation, the polymorph (III) is formed. It is thus apparent that crystalline donepezil hydrochloride monohydrate can be very sensitive to a solvent-mediated solid-solid transition into other polymorphic forms.

As donepezil hydrochloride monohydrate exhibits valuable properties for its use in pharmaceutical compositions, particularly in that it is chemically stable, is not hygroscopic, and it is well compatible with pharmaceutical excipients, it would be desirable to solve the problem of its sensitivity against the solid-solid transformation into undesired polymorphs. In particular, this need is clear in the context of large scale preparations as it is almost not possible to filter all of the precipitated solid within 10 minutes on such a scale.

### SUMMARY OF THE INVENTION

The present invention is based on the discovery that crystalline donepezil hydrochloride monohydrate can be stabilized against polymorphic modification by the use of a C5-C8 hydrocarbon compound. Accordingly, a first aspect of the invention relates to a composition comprising 100 parts by weight of crystalline donepezil hydrochloride monohydrate and 10 to 300 parts by weight of liquid, wherein at least 95% by volume of said liquid is one or more C5-C8 hydrocarbon compounds. The hydrocarbon compounds are typically selected from hexane, heptane, cyclohexane, benzene, toluene, and mixtures thereof. By surrounding the crystalline donepezil hydrochloride monohydrate with the hydrocarbon compound, the solid can be isolated and dried with a reduced risk of polymorphic conversion.

Another aspect of the invention relates to a process for isolating crystalline donepezil hydrochloride monohydrate, which comprises:

(a) precipitating crystalline donepezil hydrochloride monohydrate from a liquid crystallization media;

(b) replacing said liquid crystallization media with a second liquid media which comprises at least 95% by volume of one or more C5-C8 hydrocarbon compounds; and

(c) isolating said crystalline donepezil hydrochloride monohydrate from said second liquid media as a substantially dry solid.

The precipitation can be the initial crystallization of the donepezil hydrochloride monohydrate after the formation of the donepezil hydrochloride salt in the media or a recrystallization of donepezil hydrochloride. The liquid crystallization media is not particularly limited and includes any of the media mentioned in the prior art such as an aliphatic alcohol, tetrahydrofuran or acetonitrile, each optionally in an admixture with an aliphatic ether and/or aliphatic ester. The typical hydrocarbon compounds are as described above. The replacement can be carried out by a variety of techniques, including by filtering the crystals and washing the filtrate with the hydrocarbon compound(s) to replace the remaining liquid crystallization media in the wet filter cake.

A further aspect of the invention relates to a process, which comprises precipitating crystalline donepezil hydrochloride monohydrate from a liquid crystallization media wherein said liquid crystallization media comprises as a majority, by volume, an alcohol-miscible organic solvent and not more than 30% by volume of an aliphatic alcohol. Typically the alcohol-miscible organic solvent is ethyl acetate and the alcohol is methanol. The amount of alcohol is usually less than 20%. The process is especially useful in forming crystalline donepezil hydrochloride monohydrate in the first instance. For example, crystalline donepezil hydrochloride monohydrate can be advantageously formed by a process which comprises:

(a) dissolving a donepezil base in an alcohol-miscible organic solvent to form a donepezil solution;

(b) contacting said donepezil solution, preferably at a temperature not exceeding 20° C., with 1.0-1.3 molar equivalents of aqueous hydrochloric acid dissolved in an aliphatic alcohol, to form donepezil hydrochloride in a liquid crystallization media, wherein the amount of said aliphatic alcohol in said crystallization media is not more than 30% by volume; and

(c) precipitating crystalline donepezil hydrochloride monohydrate from said liquid crystallization media.

The crystalline donepezil hydrochloride monohydrate thus formed is preferably isolated via the use of a C5-C8 hydrocarbon compound(s) as briefly described above and in more detail below.

### DETAILED DESCRIPTION OF THE INVENTION

The present invention is based on a surprising finding that the main contribution to the reported solvent-mediated polymorphic transformation of donepezil hydrochloride monohydrate is not merely due to the lag time between precipitation and filtration, but that the transformation process into an undesired polymorph may proceed also during the filtration step and during the drying step. In short, as long as the crystals of the polymorph (I) are in contact with a liquid that can mediate the solid-solid transformation, particularly the alcohol solvent, there exists a potential for the polymorphic transformation. The most important medium, in which such a conversion may take place, is the wet cake of crystalline donepezil hydrochloride monohydrate that is subjected to filtration and, particularly, to drying. While the lag time between the precipitation of crystalline donepezil hydrochloride monohydrate and its filtration may be made as short as possible and may be adequately controlled, the time of filtration and drying is, particularly in industrial scale, incomparably longer and difficult to predict and/or control. Furthermore, the temperature necessary for effective drying of the wet cake that still comprises the liquid is usually higher than that during precipitation and filtration.

Based on this finding, the present invention serves to replace the crystallization media, to which the product is generally sensitive, with an inert medium as soon as practical after crystallization of the donepezil hydrochloride monohydrate. A suitable inert medium is a C5-C8 hydrocarbon compound, such as hexane, heptane, cyclohexane, benzene, toluene, and mixtures thereof.

Accordingly, a process for isolating crystalline donepezil hydrochloride monohydrate begins with precipitating crystalline donepezil hydrochloride monohydrate from a liquid crystallization media. The “liquid crystallization media” includes any solvent or solvent combination that dissolves either donepezil base and/or donepezil hydrochloride and which under precipitation conditions allows for the donepezil hydrochloride contained or formed therein to be crystallized as the monohydrate, i.e. Form (1). In accordance with the already known processes disclosed for providing crystalline donepezil hydrochloride monohydrate, and with obvious variations thereof, the “liquid crystallization medium” generally comprises an aliphatic alcohol such as methanol or ethanol, tetrahydrofuran or acetonitrile, wherein the aliphatic alcohol may be further admixed with an aliphatic ether, such as diethyl ether, diisopropyl ether or methyl t-butyl ether, with an ester such as ethyl acetate. Such organic solvents, especially methanol and ethanol are believed to facilitate a polymorphic modification of the crystalline donepezil hydrochloride monohydrate.

The precipitation of the crystalline donepezil hydrochloride monohydrate can be carried out in various ways. For example, the precipitation can be a recrystallization of any donepezil hydrochloride or a crystallization based on the synthesis of the salt. Several processes are described more fully below.

In a first process, donepezil hydrochloride, which has been provided by any of the known processes of the art (e.g. crude donepezil hydrochloride, the crystalline donepezil hydrochloride Form I-V or an amorphous donepezil hydrochloride), is dissolved in the liquid crystallization medium, preferably at enhanced temperature, and the crystalline donepezil hydrochloride monohydrate is crystallized/precipitated from the liquid media. Such precipitation may be obtained by, e.g., cooling the solution, adding seeding crystals, reducing the volume of the solvent, adding a contrasolvent and/or by combination of these techniques. Examples of such a type of the crystallization/precipitation process leading to the donepezil hydrochloride monohydrate are provided in the above-cited prior patents; but the process is not limited thereto.

In a second process, donepezil base is dissolved or suspended in a suitable liquid, e.g. a liquid crystallization media, and is contacted with an equivalent or greater amount of hydrogen chloride or hydrochloric acid. The donepezil hydrochloride monohydrate precipitates either spontaneously at the conditions of contacting both reagents, or the precipitation may be induced by cooling the solution, adding seeding crystals, reducing the volume of the solvent, adding a contrasolvent and/or by combination of these techniques. Examples of such a type of the salt forming process leading to the donepezil hydrochloride monohydrate are provided in the above-cited prior patents, but this process is not limited thereto.

In a third process, donepezil or donepezil hydrochloride is provided by a chemical reaction in a suitable solvent. The solution/suspension comprising the in-situ formed donepezil or donepezil hydrochloride (the reaction mixture) is then treated essentially as disclosed as in the first and the second process, respectively. The processes to obtain donepezil and/or donepezil hydrochloride by a chemical reaction are known in the art such as in the above-cited patents.

In any of the processes, care is to be taken that the medium comprises a sufficient amount of water, i.e. at least an equimolar amount of water in respect to donepezil. For example, conventional ethanol contains about 4.4% water, unless it is specially treated to break the azeotrope and remove all of the water. In this case, such a water-free ethanol, by itself and without any other water present or added to the solution or mixture would be problematic for forming the monohydrate.

It is advantageous that the slurry composition of donepezil hydrochloride monohydrate in the crystallization liquid is provided and kept at temperatures not exceeding the ambient temperature. This means that any of the three processes disclosed above should be advantageously performed in such a way that the crystals of donepezil hydrochloride monohydrate are not formed at a temperature higher than the ambient temperature, and the formed slurry of crystals are kept cool before the next step. Advantageously, the crystals should be formed and kept at temperatures of between −20 and +20 degrees Celsius.

In this respect, it is preferred that the slurry of the donepezil hydrochloride monohydrate in the crystallization solvent is provided by contacting the donepezil base with hydrochloric acid, as such process may be well kept at the desired low temperature and the salt-forming and precipitation temperature may be well controlled. The hydrochloric acid (i.e., an aqueous solution of hydrogen chloride) also provides the sufficient amount of water necessary for forming the monohydrate. It is further preferred that this salt-forming process is performed in the preferred methanol/ethyl acetate solvent system described below.

Once the crystalline donepezil hydrochloride monohydrate has been precipitated, the liquid crystallization media has done its job, namely providing a media from which the desired solid crystalline salt could be formed. The liquid crystallization media can now be replaced with a second liquid media in order to prevent or reduce polymorphic changes/disruptions. According to this aspect of the invention, the crystallization solvent included in the solid-liquid composition or slurry is replaced by the hydrocarbon liquid. The hydrocarbon liquid is, within the present invention, a C5-C8 aliphatic hydrocarbon, e.g. hexane, heptane, cyclohexane, an aromatic hydrocarbon such as benzene or toluene, or mixtures thereof, e.g. petroleum ether. The hydrocarbon liquid has the particular advantage that it does not mediate solid-solid transformation of donepezil hydrochloride monohydrate into another undesired polymorph. Preferably the hydrocarbon solvent is a low boiling volatile solvent such as n-hexane as such solvent may be removed by drying easily in subsequent isolation.

The replacement can be carried out by any suitable process until at least 95% by volume, preferably at least 99%, of the liquid of the solid-liquid composition is C5-C8 hydrocarbon compound(s). Note that “replacement” does not require that an identical amount of solvent be present in the resulting solid liquid composition or slurry as was originally present. Rather, replacement refers to the change in solvent such that the above liquid concentration is achieved. For example, the initial liquid could be drained from the vessel containing the solid-liquid composition while the second liquid media containing at least one C5-C8 hydrocarbon is added thereto until the desired concentration is reached. Usually, however, the replacement of the liquid crystallization media comprises first reducing the amount of liquid crystallization media in the liquid-solvent composition by physically separating liquid from solid. The separation is not perfect. Rather, the result is a “concentrated” liquid-solid composition having much less liquid crystallization media than before. Typically this is accomplished by filtering or centrifugation, although decanting could also be used, etc. The point is to reduce the amount of liquid in the solid liquid composition. To the concentrated crystalline donepezil hydrochloride monohydrate composition, such as a wet filer cake, is then added the second liquid media. The addition can be by washing, one or more times and especially on the filter, the concentrated crystalline donepezil hydrochloride monohydrate with the C5-C8 hydrocarbon to thereby replace the liquid crystallization media with the inert second liquid media. Alternatively, the concentrated crystalline donepezil hydrochloride monohydrate can be dispersed in the second liquid media to obtain the desired concentration. Typically such a re-slurrying of the wet product in the hydrocarbon solvent is followed by an additional separation process. In any event, the replacement is carried out until at least 95% and typically at least 99% of the liquid, by volume, contained in the composition is the C5-C8 hydrocarbon. The composition of the liquid still present in the concentrated slurry or wet cake may be monitored by ordinary methods, e.g. by analysis of the mother liquors after filtration by GC or by refractometry.

A particular aspect of the present invention relates to a solid-liquid composition that contains 100 parts by weight of crystalline donepezil hydrochloride monohydrate and 10 to 300 parts by weight of liquid, wherein at least 95% by volume, and typically at least 99% by volume, of the liquid is one or more C5-C8 hydrocarbon compounds. Such a composition contains the second liquid media and thus is protected from polymorphic modification. Further, it is concentrated, e.g. relatively low amounts of liquid, thus making is advantageous for efficient isolation of the crystals such as by heating and drying. That is, because relatively low amounts of liquid are present in the solid-liquid composition, in comparison to the usual amount of liquid immediately after precipitation, less energy is needed to dry and/or isolate the crystalline donepezil hydrochloride monohydrate from the composition. Such a composition can be formed during the replacement step and/or during the isolating step, especially as wet filter cake, but is not limited thereto.

Once protected by the relatively inert second liquid media, the crystals can be isolated with reduced risk of polymorphic change. The isolation can be carried out by any conventional method such as filtering, evaporating, and/or drying.

The isolated crystalline donepezil hydrochloride monohydrate can be dried to a substantially dry state, typically less than 1%, more preferably less than 0.5% liquid. The drying temperature preferably does not exceed 60° C. and typically is within the range of 20° C. to 40° C.

In addition to the above process for limiting the conversion of the crystalline donepezil hydrochloride monohydrate during recovery and drying, a new crystallization medium for providing the slurry of donepezil hydrochloride monohydrate with reduced polymorphic complications has also been discovered. In general, the crystallization media comprises a majority of an alcohol-miscible organic solvent, such as ethyl acetate, and an aliphatic alcohol such as methanol or ethanol, wherein the relative content of aliphatic alcohol is less than 30%, preferably less than 20% of the total mixture volume. Such medium has itself a low potential for a liquid-mediated polymorphic transformation, thereby increasing the allowable lag time before filtration of the slurry, and may be, if desired, further easily and efficiently replaced by an inert liquid before drying.

In a preferred embodiment, donepezil base is dissolved in a suitable amount of ethyl acetate, and the solution is cooled to −20 to 20° C., preferably to −10 to 10° C. At this temperature, 1.0 to 1.3 molar amount of aqueous hydrochloric acid is added. Preferably, the hydrochloric acid is added as diluted by a water- and ethyl acetate miscible diluent, for instance by an aliphatic alcohol such as methanol. The amount of the diluent is adjusted in such a way that its final amount, in respect to ethyl acetate, does not exceed 30% and preferably does not exceed 20% (v/v) of the total volume. The temperature during the contact between donepezil and hydrochloric acid should be kept at the same temperature as above. After completing the reaction, the mixture is optionally seeded with the Form I crystals and allowed to precipitate, preferably at about −10° C., whereby the suspension may be stirred for at least 30 minutes prior to filtration. If desired, the second liquid media of a C5-C8 hydrocarbon can be used to replace the crystallization media as described above.

This process allows the formation of the donepezil hydrochloride monohydrate crystals free form other polymorphic forms, under well controlled, reproducible and easy to scale up conditions, with a sufficient lag time between forming the crystals and filtration.

In a small scale (few grams), where a rapid filtration and drying may be assured, the formed crystals may be isolated just by ordinary filtration and drying. In a bigger scale, the process should typically be modified as disclosed above. For example, the formed crystals are filtered off, the wet cake is washed first with ethyl acetate (to remove the rests of methanol and water) and then with the hydrocarbon, preferably with n-hexane or n-heptane. The filtration and washing should be preferably performed at temperatures close to 0° C. or lower. Wet product may be dried under ordinary conditions, preferably at temperatures not exceeding 40° C.

The invention having been explained above, it will be further illustrated with reference to the following non-limiting examples.

### EXAMPLE 1

In a 2-litre three necked flask equipped with a mechanical stirrer, 100 g of donepezil was dissolved in 1000 ml of ethyl acetate at ambient temperature. The solution was cooled to −8° C. 23 ml of 37% hydrochloric acid was dissolved in 170 ml of methanol and cooled to −10° C. This solution was added to the stirred solution of donepezil in ethyl acetate. No exothermic effect was observed. After 2 minutes of stirring, the solution was seeded with 100 mg of donepezil hydrochloride monohydrate (Form I). Crystallization started, a small exothermic effect was observed (the temperature raised to −6° C.). The resulting suspension was stirred at −6 to −8° C. for 45 minutes.

The solid was isolated by filtration on a 1 liter glass-filter which was cooled at 4° C. The wet cake was washed with cold 150 ml of ethyl acetate (0° C.) and 2×100 ml of cold n-heptane. The wet solid was spread out on a glass dish and allowed to dry at ambient conditions and mixed regularly.

The weight of the wet solid was monitored.

| | |
|---|---|
| Start of drying: | 181 g |
| After 1 hour: | 144 g |
| After 2 hours: | 104 g |
| After 18 hours: | 99.4 g |
| Yield: | 99.4 g |

XRPD showed pure FORM I (monohydrate)

### EXAMPLE 2

In a 3-liter three necked flask equipped with a mechanical stirrer, 150 g of donepezil was dissolved in 1500 ml of ethyl acetate at ambient temperature. The solution was cooled to −8° C. 34.5 ml of 37% hydrochloric acid (1.05 eq.) was dissolved in 255 ml of methanol and cooled to −10° C. This solution was added to the stirred solution of donepezil in ethyl acetate. No exothermic effect was observed. After 2 minutes of stirring, the solution was seeded with 100 mg of donepezil hydrochloride monohydrate (Form I). Crystallization started, a small exothermic effect was observed (the temperature raised to −6° C.). The resulting suspension was stirred at −6 to −8° C. for 45 minutes. The solid was isolated by filtration on a 1 liter glass-filter which was cooled at 4° C. The wet cake was washed with cold 225 ml of ethyl acetate (0° C.) and 2×150 ml of cold n-heptane

The wet solid was spread out on a glass dish and allowed to dry at ambient conditions and mixed regularly for 24 hours.
Yield: 157.0 g
XRPD showed pure FORM I (monohydrate)

Each of the patents mentioned above are incorporated herein by reference. The invention having been described it will be obvious that the same may be varied in many ways and all such modifications are contemplated as being within the scope of the invention as defined by the following claims.

I claim:

1. A composition comprising 100 parts by weight of crystalline donepezil hydrochloride monohydrate and 10 to 300 parts by weight of liquid, wherein at least 95% by volume of said liquid is one or more C5-C8 hydrocarbon compound.

2. The composition according to claim 1, wherein said C5-C8 hydrocarbon compound is selected from the group consisting of hexane, heptane, cyclohexane, benzene, toluene, and mixtures thereof.

3. The composition according to claim 2, wherein said hydrocarbon comprises at least 99% by volume of said liquid.

4. A process for isolating crystalline donepezil hydrochloride monohydrate, which comprises:
(a) precipitating crystalline donepezil hydrochloride monohydrate from a liquid crystallization media;
(b) replacing said liquid crystallization media with a second liquid media which comprises at least 95% by volume of one or more C5-C8 hydrocarbon compounds; and
(c) isolating said crystalline donepezil hydrochloride monohydrate from said second liquid media as a substantially dry solid.

5. The process according to claim 4, wherein said replacing step comprises reducing the amount of liquid crystallization media by a physical separation process to form a concentrated crystalline donepezil hydrochloride monohydrate composition and adding said second liquid media to said concentrated composition.

6. The process according to claim 5, wherein said addition of said second liquid media comprises washing said concentrated composition one or more times with said second liquid media.

7. The process according to claim 6, wherein said concentrated composition is washed on a filter.

8. The process according to claim 5, wherein said addition of said second liquid media comprises dispersing said concentrated composition in said second liquid media one or more times.

9. The process according to claim 5, wherein said physical separation comprises filtering said crystalline donepezil hydrochloride monohydrate.

10. The process according to claim 4, wherein said isolating step comprises drying off said stable liquid media at a temperature not greater than 40° C.

11. The process according to claim 10, wherein said substantially dry solid crystalline donepezil hydrochloride monohydrate contains less than 0.5% liquid by weight.

12. The process according to claim 4, wherein said liquid crystallization media comprises an aliphatic alcohol, tetrahydrofuran or acetonitrile, optionally in an admixture with an aliphatic ether and/or with an aliphatic ester.

13. The process according to claim 12, wherein said liquid crystallization media comprises an aliphatic alcohol selected from methanol and ethanol.

14. The process according to claim 4, wherein said second liquid media comprises hexane, heptane, cyclohexane, benzene, toluene, or mixtures thereof.

15. A process, which comprises precipitating crystalline donepezil hydrochloride monohydrate from a liquid crystallization media wherein said liquid crystallization media comprises as a majority, by volume, an alcohol-miscible organic solvent and not more than 30% by volume of an aliphatic alcohol.

16. A process for making crystalline donepezil hydrochloride monohydrate, which comprises:
(a) dissolving a donepezil base in an alcohol-miscible organic solvent to form a donepezil solution;
(b) contacting said donepezil solution at a temperature not exceeding 20° C. with 1.0-1.3 molar equivalents of aqueous hydrochloric acid dissolved in an aliphatic alcohol, to form donepezil hydrochloride in a liquid crystallization media, wherein the amount of said aliphatic alcohol in said crystallization media is not more than 30% by volume; and
(c) precipitating crystalline donepezil hydrochloride monohydrate from said liquid crystallization media.

17. The process according to claim 16, wherein said alcohol-miscible organic solvent is ethyl acetate and said aliphatic alcohol is methanol.

18. The process according to claim 17, wherein said methanol does not exceed 20% by volume of said liquid crystallization media.

19. The process according to claim 16, which further comprises:
reducing the amount of liquid crystallization media by a physical separation process to form a concentrated crystalline donepezil hydrochloride monohydrate composition;
replacing said liquid crystallization media with a second liquid media which contains at least 95% by volume of one or more C5-C8 hydrocarbon compounds; and
drying said crystalline donepezil hydrochloride monohydrate as a substantially dry solid.
